(19) United States
(12) Patent Application Publication
(10) Pub. No.: US 2026/0053649 A1
(43) Pub. Date: Feb. 26, 2026

(54) INTRALUMINAL DEVICES FOR TREATING BENIGN PROSTATIC HYPERPLASIA

(71) Applicant: Rivermark Medical, Inc., Milwaukee, WI (US)

(72) Inventors: Maurino Galinato Flora, San Jose, CA (US); Anand Doraiswamy, San Francisco, CA (US)

(21) Appl. No.: 19/373,605

(22) Filed: Oct. 29, 2025

Related U.S. Application Data
(63) Continuation of application No. 16/972,221.
(60) Provisional application No. 62/682,001, filed on Jun. 7, 2018.
(60) Provisional application No. 62/789,876, filed on Jan. 8, 2019.

(51) Int. Cl. A61F 2/89; A61F 2/848; A61F 2/966

(52) U.S. Cl. CPC A61F 2/89; A61F 2/848; A61F 2/966; A61F 2002/8483; A61F 2002/9665; A61F 2210/0014; A61F 2250/0039

(57) ABSTRACT

Some embodiments of the invention advantageously leverage the expansion of the prostatic urethra using adjustable lumen tension rings (ALTRs) of desired sizes to control and improve flow throughout the range of the urethra. As such, some embodiments include tension ring(s) or cylinders that sits either inside or outside the prostatic wall and is at least partially within the lumen and/or is partially or fully anchored, attached, adhered, or otherwise held in place with respect to the bladder or other locations within the urethra and/or elsewhere in the urethra. The expansion of the lumen can be pre-configured based on pre-operative metrology of the prostatic urethra to a customized and adjustable fit across the various zones within the urethra and based on the patient specific and evolving needs.

### PRIORITY CLAIM

This application is a continuation of U.S. Application No. 16/972221 filed on Dec. 4, 2020, which is a the U.S. national phase entry under 35 U.S. C. § 371 of International Application No. PCT/US2019/036162 filed on Jun. 7, 2019, which claims the benefit under 35 U.S. C. § 119(e) of U.S. Provisional No. 62/682,001 filed on Jun. 7, 2018 and U.S. Provisional No. 62/789,876 filed on Jan. 8, 2019, each of the foregoing of which are hereby incorporated by reference in their entireties.

### BACKGROUND

Benign prostatic hyperplasia (BPH) is a common benign condition that develops in men and is bothersome in elderly patients. In this condition, the prostate gland is enlarged and not cancerous. Benign prostatic hyperplasia is also called benign prostatic hypertrophy or benign prostatic obstruction.

The prostate gland is a fibromuscular and glandular organ lying just inferior to the bladder. As the prostate enlarges, the gland presses against and pinches the prostatic urethra. This leads to weakening the bladder and ability to completely empty the bladder. The narrowing of the prostatic urethra and urinary retention causes the symptoms observed with BPH. As many as 14 million men in the United States have lower UTI symptom, suggestive of BPH. Approximately half of all men over the age of 50 will develop an enlarged prostate. By the time men reach their 70's and 80's, approximately 85-90% of them will experience urinary symptoms from BPH.

While the etiology is not completely well-understood, it is thought to be multifactorial and endocrine controlled. BPH develops in the transitional zone of the prostatic urethra. Symptoms often include irritative and obstructive flow. Specifically, the following symptoms may be suggestive of BPH: urinary frequency, urinary urgency, trouble starting a urinary stream, retention, incontinence, nocturia, pain after ejaculation. Complications of BPH include bladder stone, urinary tract infection, hematuria, bladder decompensation, renal failure, and acute/chronic urine retention.

Pharmacologic approaches for treatment include use of alpha blockers such as phenoxybenzamine (non-selective), prazosin (short-acting), terazosin & doxazosin (long-acting), and tamsulosin, alfuzosin, and silodosin (α1a) selective blockers. Additionally, 5α-reductase inhibitors and combination therapies are also used. Pharmacologic approaches are inadequate in the effectiveness and often used as short-term treatments. Side-effects of pharmacologic approach include orthostatic hypotension, dizziness, tiredness, retrograde ejaculation, rhinitis, and headache.

Conventional and recent surgical therapies include a) Transurethral resection of the prostate (TURP), b) Transurethral incision of the prostate (TUIP), c) LASER therapy, d) Other forms of energies to vaporize the prostate, d) Balloon dilation, e) Prostatic stents, and f) Urolift procedure. With TURP, 90% of prostatectomy is done endoscopically to alleviate symptoms and improve flow rate. However, TURP requires spinal or general anesthesia and a 4-6 week recovery time with catheterization. Additionally, some complications include impotence, incontinence, bleeding, retrograde ejaculation, and TUR syndrome (vomiting, nausea, confusion, hypertension, etc.). With TURP, open prostatectomy is performed when prostate gland is over 100 gms or when BPH occurs with vesical stone. With c) LASER therapy, LASER energy is used to ablate prostate which has advantages in minimal blood loss, ability to be done as out-patient procedure. However, LASER therapy requires longer post-operative catheterization time and requires high cost of LASER fiber and generators. Other forms of energies including microwave, focused ultrasound, water-induced thermotherapy, electrovaporization, etc. have also been tried with generally poor outcomes. Transurethral balloon dilation of the prostate has also been tried in the past with poor outcomes. Prostatic stents (temporary and permanent) have also been employed in the past. Poor anchoring, migration of the stent, and difficult removal have led to poor outcomes/utilization. Recent developments include Urolift—a technique where the prostates are tied away from the urethra. While it is minimally invasive, the procedure still has some disadvantages such as use of a temporary catheter, leaks, and the relative invasiveness of manipulating the prostate.

### SUMMARY

Some embodiments of the invention are directed to minimally invasive systems and methods for maintaining a patency of a body lumen. One non-limiting indication is treating benign prostatic hyperplasia (BPH). In some embodiments, one, two, or more adjustable shape memory lumen tension rings (or springs) can be configured to be placed within the lumen of the prostatic urethra or another body lumen. The tension ring(s) exert a radially outward mechanical force on the prostatic urethral lumen to restore the patency of the prostatic urethra and improve urine flow therethrough, typically although not exclusively in a non-penetrating fashion. The rings can include a proximal end, distal end, and a coiled section comprising a plurality of revolutions therebetween, and proximal and distal eyelets for ease in manipulation, relocation, or retraction using a separate retrieving device. The coiled section has a variable outer diameter along its length. The rings can be made of a small diameter shape memory wire (e.g., about 0.10″), such as nitinol. The relatively thin wire combined with the radial spring force of the ring advantageously reduces the risk of undesirable encrustation/crystallization with urine as a majority of the wire is embedded in tissue of the luminal wall and does not contact the urine flow. The device can be coated with PTFE, silicone, and/or other hydrophilic and/or hydrophobic coating materials. In some embodiments, a device can be coated with one or more therapeutic agents, including drugs such as alpha-1 blockers, 5 alpha-reductase inhibitors and combination therapies, such as in an extended-release coating. In some embodiments, a device is not coated with one or more therapeutic agents, such as a drug.

Some embodiments of the invention advantageously leverage the expansion of the prostatic urethra using devices including adjustable lumen tension rings (ALTRs) of desired sizes to control and improve flow throughout the range of the urethra. As such, some embodiments include a adjustable tension ring(s) or cylinders that sits at least partially within the lumen and/or is partially or fully anchored, attached, adhered, or otherwise held in place with respect to the bladder or other locations within the urethra and/or elsewhere in the urethra. The expansion of the lumen can be configured to change at the various zones within the urethra independently and based on the patient specific needs. In some embodiments, the tension rings may be 1, 2, 3, 4, 5, 6 separate rings of various sizes. The forces of the prostate against the prostatic wall can be translated to various tension ring sizes for the various locations along the length of the wall. In some embodiments, the system can be configured to control the flow rate of urine through the urethra. In some embodiments, the rings can be substituted by cylinders, including some with fixation elements. The devices, e.g., Adjustable Lumen Tension Rings can be configured to fit a specific patient's range in urethral dimensions in some embodiments.

In some embodiments, the devices, e.g., adjustable lumen tension rings (ALTRs) could include one, two, or more fixation elements. The fixation elements can promote fixation of the ALTR to the urethral wall. In some embodiments, the ALTR may include a protrusion or indentation for stabilizing and/or fixing the ALTR at the wall. The fixation elements can also include sub-elements to anchor the ALTR to the wall, for example, grooves, teeth, ridges, or a saw-tooth pattern, for example. In some embodiments, two or more of the same or different fixation elements can be used in combination.

In some embodiments, the devices, e.g., ALTR can include one or many features such as grooves or loops to allow easy capture and removal of the ALTR, if/when needed.

Also disclosed herein are various materials for the device, e.g., ALTR including: shape-memory alloy (SMAs), flexible metals such as stainless steel, titanium, etc. and flexible polymers including shape memory polymers (SMPs) that can be implanted through a small incision and spring back to the original configuration without damage. In some embodiments, the ALTR material may include coatings to prevent degradation and encrustation. The coating might be of hydrophobic or hydrophilic in nature such as silicone. In some embodiments, the coating could include PTFE or ePTFE. In some embodiments, the coating could include flexible silicones, hydrogels, mucoadhesive substrate, pressure-sensitive adhesives, and other suitable elastomers, such as synthetic rubbers. In one or more embodiments, a coating having a micropattern may include and/or be formed from a biologically-derived protein structure (e.g., collagen, etc.)

In some embodiments, disclosed is a method of surgically implanting the ALTR. An image-guided flexible cystoscope or a catheter with a camera can be utilized in combination with a trigger mechanism to deploy the ALTR with each click. The ALTR can be preloaded for the various sizes into a cartridge which can be attached to the flexible cystoscope. The advantage of such a technique is to accurately position the ALTR from the bladder to deploy in the appropriate zones within the prostatic urethra. The ALTR can be implanted via a placement tool allowing use of the manipulating features which can also be used to easily relocate or retract in some cases. The ALTR could be implanted alone, or in combination with other ALTRs in some embodiments.

The ALTRs can be customized for a specific patient, including age, race, demographic, predispositions, urethral dimensions, prostatic dimensions, anatomical differences, and other factors unique to the patient. In some embodiments, a device can be deployed to a vascular, or non-vascular body lumen. Non-vascular body lumens may have only intermittent rather than constant flow of body fluids therein in some cases.

In some embodiments, a device can include any combination of the following features, or others as disclosed elsewhere herein:
1. A device, such as a Adjustable Tension Ring (ALTR) embodiment stored and loaded into a delivery system, once deployed the stored embodiment will spring and take shape with its shape memory and form a tension or torsion ring or rings that is inside or outside the urethral wall and is larger than the urethral lumen diameter, providing separation between the previously compressed lumen of the urethra in the body of any animal. One or more embodiments with varying sizes can be deployed in the compressed prostatic urethra at various locations. The embodiments can be a combination of smaller diameter at the ends (to prevent leakage) and larger diameter in the middle (to anchor and prevent migration) or the same ALTR can contain any number of these variations.
2. The device of Example 1, made of round/rectangular/square Nitinol shape-memory alloy (NiTi) wire/tubing. The wire tubing OD can be, for example, between about 0.001″ and about 0.20″. The non-circular wire embodiment can be between 0.001″-2.00″ by 0.001″-2.00″ In some embodiments.
3. The device of any of Example 1 or 2, comprising various perforations and extensions to anchor to the lumen wall.
4. The device of any of the previous examples, comprising a plurality of perforations or features to allow rapid exchange, repositioning, and/or removal.
5. The device of any of the previous examples, designed and selected based on patient specific anatomy through pre-operating imaging.
6. The device of any of the previous examples, designed with irregularities on the peripheral circumference including ridges, indentations, etc. to allow better anchoring and preventing migration.
7. The device of any of the previous examples, implanted in the same operative procedure as positioning another device in the prostatic urethra of the patient.

8. The device of any of the previous examples, designed to vary the sweep angle between about 10 and about 360 degrees. Additionally, the ALTRs may have multiple continuous or dis-continuous sweeps from 1 to 10, or more.
9. The device of any of the previous examples, may be used in multiples and with various sizing within the urethra to control the shape of the expansion and amount and direction flow.
10. The device of any of the previous examples, where the device can be customized for a specific patient or animal, including age, race, demographic, genetic predispositions, urethral dimensions, prostatic dimensions, anatomical differences, and other factors unique to the patient or animal.
11. The device of any of the previous examples, where the device is a tension ring which can be customized to shrink and/or lengthen upon adjusting the manipulating feature of the device, for safe and easy relocation, repositioning or removal.
12. A device, e.g., Adjustable Tension Ring (ALTR) embodiment stored and loaded into a delivery system, once deployed the stored embodiment will spring and take shape with its shape memory and form a tension or torsion ring or rings that is inside or outside the urethral wall and is larger than the urethral lumen diameter, providing separation between the previously compressed lumen of the urethra in the body of any animal. A balloon catheter is deployed prior to the deployment of the ALTR to allow enlargement of the prostatic urethra allowing the ALTR to be deployed with ease.
13. The device of any of the previous examples, where the ALTR has a sharp leading edge that can pierce the urethral wall to anchor into the prostate itself and keep it expanded from outside the urethral wall.
14. The device of any of the previous examples, where the ALTR has a manipulating feature that is anchored inside the urethral wall to allow subsequent manipulating of the ALTR.
15. The device of any of the previous examples, where the ALTR can be delivered in a shrunken (or smaller) state by manipulating temperature of the ALTR (e.g. Nitinol) using external energy.
16. A device, e.g., Adjustable Tension Ring (ALTR) embodiment that can be manipulated by an insertion tool whose temperature can be externally controlled through an energy source (electrical, mechanical, thermal, RF, etc.), such that it can alter the shape (shrink or expand) of the ALTR to make insertion or retrieval procedure both minimally invasive, responsive, and easy to manipulate/handle.
17. A device, e.g., ALTR delivery system comprising an extruded metal or plastic tubing whereby the ALTR is in a stored state, and once the ALTR is deployed to the desired location, the ALTR takes shape with its shaped memory and configuration.
18. A device, e.g., ALTR delivery system comprising an extruded metal or plastic tubing whereby the extruded metal or plastic tubing is printed with measurement markers to serve as a reference point in ALTR deployment.
19. A device, e.g., ALTR delivery system with an expandable member, such as a balloon at the distal end which can facilitate opening of the prostatic urethra and anchoring from the bladder opening to accurately deploy and position the ALTR.
20. The device, e.g., ALTR delivery system can be mechanical wherein a user can feel the incremental advancement of the ALTR towards the distal shaft as guided by the cystoscope. The deployment mechanism can be conveyed from the handle of the device. The deployment mechanism can be geared so that the advancement is measured based on the predetermined measured location.
21. The device, e.g., ALTR delivery system can also be a stapler type whereby the ALTR is stored in a cartridge containing a predetermined count such as, for example, 1 to 6 ALTRs. The ALTRs can be individually dispensed from the cartridge via a trigger mechanism from the handle of the ALTR delivery system.
22. The device, e.g., ALTR delivery system may have the ALTRs pre-loaded into multiple cartridges allowing deployment of all ALTRs in one single procedure minimizing time needed.
23. The device, e.g., ALTR delivery system can be in conjunction with the cystoscope via a catheter delivery system place adjacent (alongside the cystoscope or delivery post cystoscope visualization via predetermined length noted in the cystoscope).
24. The device, e.g., ALTR delivery system can be either manually via pusher catheter-shaft/tubing; or mechanically driven such as with staples; or electromechanically delivered into the desired location; and or energy driven, e.g., radio frequency, ultrasonic, thermal, or electronic signal.
25. The device of any of the previous examples, that may have features such as a loop, hook, or eyelets to allow easy capture using an ALTR retrieval system that can allow the ALTR to compress and withdraw into the system in cases where repositioning or removal is desired.
26. The device of any of the previous examples, configured to be withdrawn by compression or re-folding the embodiment back into a linear or FIG. 8 shape and either fully withdrawn or repositioned.
27. The device, e.g., ALTR can be withdrawn by capturing the manipulating feature of the ALTR and re-winding into a track/guide.
28. The device, e.g., ALTR can be withdrawn by capturing the manipulating feature and shrinking the ALTR by manipulating temperature of the ALTR (e.g., Nitinol) using external energy.
29. The device, e.g., ALTR, ALTR delivery systems and ALTR retrieval systems may have polymeric coatings including fluoropolymers and silicone, etc. The coatings may also be used to seal and prevent coagulation, debris accumulation, or degradation over time.
30. The device of any of the previous examples, where it can be used for other applications within the body of the animal where manipulation of the lumen diameter is desired, either vascular and/or non-vascular. For example, devices, e.g., ALTRs can be used for coronary stent applications to allow smooth, uninterrupted, and adequate blood flow in the heart; peripheral vascular stents, neurovascular stents, other arterial or venous applications; devices can be used as ophthalmic stents to allow smooth, uninterrupted, and adequate aqueous flow to reduce intraocular pressure (IOP), such as placement in the trabecular space (e.g., Schlemm's canal), superciliary space, and/or subconjunctival space. In some embodiments, a device can be utilized for placement in the GI tract, such as the esophagus, pylorus, small or large intestine, or biliary tree, such as the common bile duct, cystic duct, pancreatic duct, or hepatic duct for example. In each these cases, the features of the ALTR allow ease of repositioning, relocation, and removal.
31. The device of any of the previous examples, where it can be delivered via a delivery system, and the ALTR is deployed longitudinally within the body of the animal where manipulation of the lumen diameter is desired. The ALTR can include 3, 4, or more sections whereby the sections are pulled apart the sections separate and the pre-shaped internal mechanism extends creating a pre-shaped profile of the ALTR. Depending on the desired shape, the sections are separated and joined at the ends via a hook and loop closure. The closure can also be sutured to complete loop.
32. The device of any of the previous examples, configured for deployment using a tool and/or mechanism to hold both sections of the ALTR align with the axis of the ALTR. After which the ALTR is repositioned by turning the ALTR perpendicular to the deployed axis.
33. The device of any of the previous examples can be of a hypotube material such as Ni-Ti (Nickel-Titanium) or Stainless-Steel (SS) example. The hypotube materials are connected via pre-formed wire such as Ni-Ti or SS and secured inside the hypotube via an epoxy glue, and or similar adhesive. Once the sections are separated, the sections form the basis of the desired ALTR shape.
34. The device of any of the previous examples can be visualized before and or after deployment and or delivery to ensure the ALTR is located. The visualization system can be done either intraluminal and or extraluminal in nature either by using optical or ultrasound or other forms of imaging and/or video to visualize the topography of the urethra.
35. The device of any of the previous examples can be deployed within the channel of the endoscope and or cystoscope wherein the endoscope and or cystoscope has an integrated visualization system. The channel can be, for example, have a diameter of between about 1 mm and about 6 mm or about 30%, 40%, 50%, 60%, 70%, or more or less of the circumference, or ranges including any two of the foregoing values.
36. The device of any of the previous examples can be deployed using a specific delivery system carrying the ALTR within a sheath. Once the sheath with the ALTR is positioned as desired, the sheath is either pulled or pushed to expose the ALTR for deployment.
37. The device of any of the previous examples with customized sizing based on biometry of the prostatic urethral space. The prostatic urethra can be measured or imaged as a pre-operative scan using various qualitative or quantitative measurement tools to determine the customized fit of ALTR size(s) required for the specific patient's need. Biometric measurements including prostatic dimensions, PUA (prostatic urethral angle), IPP (intravesical prostate protrusion), RPUL (the ratio between the prostatic urethral length and prostate volume) may be used to determine and customize the ALTR design to fit the specific physiological and anatomical need of the patient.
38. Additionally, as the patient ages and as prostate undergoes physiological changes, the device may be replaced (with other smaller or larger sizes (e.g., lengths or diameters) or tensile strength) to fit the changing need.

In some embodiments, disclosed herein is a device for maintaining patency of a prostatic urethra, that can include one or more of a shape memory member comprising a proximal end, a distal end, and a passageway therebetween configured to facilitate flow of body fluids therebetween. The shape memory member can include a plurality of partial or complete loops between the proximal end and the distal end. The shape memory member can also include a central portion and lateral portions. The central portion can include a first diameter and the lateral portions comprise a second diameter. The first diameter can be not equal to the second diameter. The shape memory member can further include a first radially compressed configuration transformable to a second radially enlarged configuration.

In some configurations, the first diameter is larger than the second diameter.

In some configurations, the first diameter is smaller than the second diameter.

In some configurations, the central portion has a generally constant first diameter throughout the entire length of the central portion.

In some configurations, the lateral portions have a generally constant second diameter throughout the entire length of the central portion.

In some configurations, non-adjacent loops of the device are only connected to each other via directly adjacent loops.

In some configurations, the shape memory member has a diameter of between about 0.001″ and about 0.020″.

In some configurations, the shape memory member has a non-circular cross section, the cross-section having a major axis and a minor axis, wherein the minor axis dimension is between about 0.001″ and about 0.020″.

In some configurations, a device further includes one or more manipulation features proximate at least one of the proximal end and the distal end.

In some configurations, the one or more manipulation features are selected from the group consisting of an eyelet, a hook, and a loop.

In some configurations, the one or more manipulation features are selected from the group consisting of an eyelet, a hook, and a loop.

In some configurations, the shape memory member comprises a hydrophobic coating.

In some configurations, the shape memory member comprises a hydrophilic coating.

In some configurations, the proximal end comprises a sharp edge configured to pierce the urethral wall to anchor into the prostate itself and keep it expanded from outside the urethral wall.

In some configurations, the shape memory member comprises surface irregularities configured to promote anchoring and/or prevent migration.

In some configurations, the surface irregularities comprise one or more of ridges, roughened surface, pores, and indentations.

In some configurations, there are between about 2 and about 30 partial or complete loops.

Also disclosed herein is a delivery system that can include a device with any number of features as disclosed herein, and a delivery tool comprising a device channel comprising an outer sheath and an inner member, the device configured to be disposed between the inner member and the outer sheath, the delivery tool comprising a locking member configured to reversibly lock the device within the device channel.

In some configurations, the delivery tool comprises a cystoscope.

Also disclosed herein is a method of treating benign prostatic hyperplasia in a patient, comprising expanding a lumen of the prostatic urethra in the patient using an expandable member; radially expanding at least one device comprising a shape memory member comprising a plurality of windings within the lumen of the prostatic urethra to expand the diameter of the prostatic urethra, the at least one device comprising a larger diameter portion and a smaller diameter portion, the larger diameter portion providing a radial force against the prostatic urethra sufficient to maintain patency of the prostatic urethra; and/or unlocking a manipulation feature of the at least one device from a delivery tool.

In some configurations, the expandable member comprises a balloon.

In some configurations, the method also comprises radially expanding a plurality of devices.

In some configurations, the plurality of devices is radially expanded sequentially.

In some configurations, the plurality of devices comprises varying sizes and/or shapes.

In some configurations, the method further comprising removing the delivery tool from the prostatic urethra.

In some configurations, the delivery tool comprises a cystoscope comprising a device channel, an outer sheath, and an inner member, wherein the device is disposed between the inner member and the outer sheath and in a radially compressed configuration during delivery.

In some configurations, the method further comprises axially moving the outer sheath relative to the device to allow the device to radially expand.

In some configurations, the device channel comprises a diameter of between about 1 mm and about 6 mm.

In some configurations, following radially expanding the at least one device within the lumen of the prostatic urethra, no more than about 25% of an entire surface area of the device is exposed to urine flow within the prostatic urethra.

In some configurations, following radially expanding the at least one device within the lumen of the prostatic urethra, no more than about 3 cm² of the entire surface area of the device is exposed to urine flow within the prostatic urethra.

In some configurations, the shape memory member has a maximum diameter of less than about 0.020″.

In some configurations, the device once implanted does not extend axially outside of the prostatic urethra.

In some configurations, the method further comprises delivering energy to the at least one device to transform the size and/or shape of the device.

In some configurations, the at least one device is custom created based on biometry of the patient's prostatic urethra.

In some configurations, the at least one device is custom created based on measured patient parameters selected from one or more of pre-operative imaging, prostatic dimensions, PUA (prostatic urethral angle), IPP (intravesical prostate protrusion), and RPUL (ratio between the prostatic urethral length and prostate volume).

Also disclosed herein is a method of repositioning or removing a device for treating benign prostatic hyperplasia in a patient, comprising delivering an effector tool proximate the prostatic urethra and a previously implanted device residing within the lumen of the prostatic urethra, wherein a previously implanted device comprises one or more shape memory member comprising a plurality of windings, the windings forming a lumen with a variable inner diameter, the device maintaining the patency of the lumen of the prostatic urethra; contacting a manipulation feature of the previously-implanted device with the effector tool; locking the manipulation feature of the at least one device; and/or removing or repositioning the device.

In some configurations, the method can further include delivering energy from the effector tool to the manipulation feature to change the size and/or shape of the device.

In some configurations, the manipulation feature comprises a hook, a loop, a magnet, or a threaded feature.

### BRIEF DESCRIPTION OF THE DRAWINGS

FIG. 1 illustrates a cross-sectional view of a male urethra and associated anatomy.

FIG. 2A shows a schematic cross-section of the prostatic urethra and the bladder in the case of a healthy prostate and an enlarged prostate. The schematic of an enlarged prostate is further detailed in FIG. 2B.

FIG. 3A illustrates a cross-sectional view of the enlarged prostate in the prostatic urethra. FIG. 3B illustrates the use of a guide catheter into the prostatic urethra. FIG. 3C illustrates the deployment of a balloon technique to expand the prostatic urethra. FIG. 3D illustrates the deployment of the ALTRs (Adjustable Lumen Tension Rings) in a minimally invasive procedure. FIG. 3E illustrates the deployment of the leading edge of the ALTR into the prostatic urethral wall into the prostate. FIG. 3F illustrates further deployment of the ALTR into the prostate while the prostate is an expanded (balloon) state. FIG. 3G illustrates the in-situ location of the ALTR with the manipulation feature(s) remaining inside the urethral wall. FIG. 3H illustrates the deflation of the balloon once the ALTR is able to anchor the prostate and keep the prostatic urethra wide-open. FIG. 3I illustrates the removal of the balloon and guide-wire and shows the ALTR expanding the prostate from outside the lumen and keeping the prostatic urethra wide-open.

FIG. 3J illustrates a retrieval device to remove the ALTR with ease and using a minimally invasive procedure. FIG. 3K illustrates the ALTR partially wound into the retrieval device. FIG. 3L illustrates the ALTR fully wound into the retrieval device.

FIGS. 3M-3O schematically illustrate additional embodiments of prosthetic devices implanted within the prostatic urethra.

FIG. 4 illustrates a variant of the ALTR device where the device is able to guide along the interstitial space between the prostate and the external urethral wall.

FIGS. 5A-C illustrates a variant of the ALTR device where the device is of a wire form. FIG. 5A is an isometric view; FIG. 5B is a side view, and FIG. 5C is a top view (entry).

FIGS. 6A-C illustrates a variant of the ALTR device where the device is of a ribbon form. FIG. 6A shows a flat ribbon. FIG. 6B shows a flat ribbon with the manipulating feature at the leading or trailing edge. FIG. 6C shows a bent ribbon.

FIGS. 6D-F illustrate additional embodiments of devices that can include no more than a single, or a plurality of, complete or incomplete rings configured to maintain the patency of a lumen.

FIG. 6G illustrates use of multiple devices of various sizes in the lumen to control the shape of the expansion and amount and direction flow.

FIGS. 6H-6I illustrates a generally cylindrical embodiment of a device, according to some embodiments.

FIGS. 6J-6M illustrate embodiments of devices using a plurality of multifilaments or polyfilaments to expand the lumen wall.

FIGS. 6N-6P illustrate embodiments of devices that are delivered in a minimally invasive form, such as a substantially linear biased form in some cases as shown, and retain the intended unbiased shape in-situ.

FIG. 7A illustrates delivery of the ALTR into the urethra with an enlarged prostate. FIG. 7B illustrates the delivered and in-situ ALTR where the tension of the ALTR is able to keep the prostatic wall expanded and open. FIG. 7C shows additional ALTRs delivered within the prostatic urethra. FIG. 7D shows a variant of the ALTR with a higher diameter in the central portion of the ALTR. FIG. 7E shows additional ALTRs of this variant delivered within the prostatic urethra. FIG. 7F shows a variant ALTR that covers the entire length of the prostatic urethra.

FIG. 7G illustrates a retrieval device to remove the ALTR with ease and using a minimally invasive procedure. FIG. 7H illustrates the ALTR partially wound into the retrieval device. FIG. 7I illustrates the ALTR fully wound into the retrieval device.

FIG. 8 illustrates a variant of the ALTR delivered inside the urethral walls.

FIGS. 9A-C illustrates a variant of the ALTR with two manipulating features on the leading and trailing ends. FIG. 9A is a top-view; FIG. 9B is a side view; and FIG. 9C is an isometric view of the ALTR.

FIGS. 10A-C illustrates a variant of the ALTR with variable diameter along the length of the ALTR. FIG. 10A is a top-view; FIG. 10B is a side view; and FIG. 10C is an isometric view of the ALTR.

FIGS. 11A-C illustrates a variant of the ALTR with a torsion spring design. FIG. 11A is a top-view; FIG. 11B is a side view; and FIG. 11C is an isometric view of the ALTR.

FIGS. 12A-C illustrate various views of an embodiment of an ALTR with a large diameter central portion and smaller diameter lateral portions.

FIGS. 13A-C illustrate various views of an embodiment of an ALTR with a small diameter central portion and larger diameter lateral portions.

FIGS. 14A-D illustrate various views on an embodiment of an ALTR reversibly coupled to a delivery tool.

FIGS. 15-16 illustrate published information on clinical parameters based on prostatic urethral biometry (PUA, IPP, and RPUL, etc.) and correlation to clinical signs and symptoms of BPH.

### DETAILED DESCRIPTION

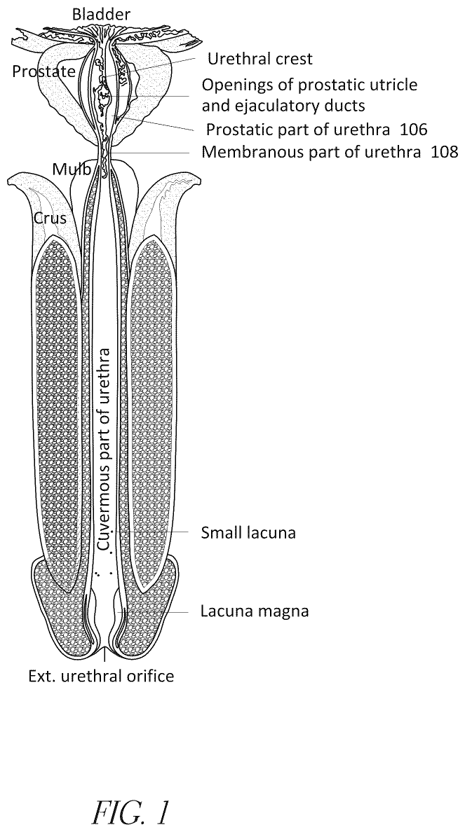

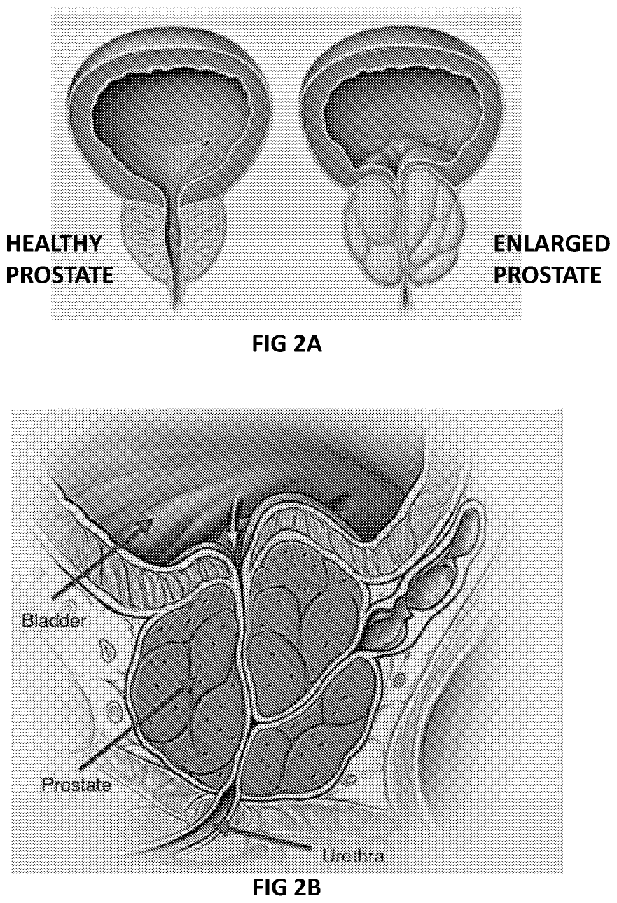

Several factors influence the onset and progression of BPH (Benign Prostate Hyperplasia, also known as Benign Prostate Hypertrophy). The most common factor is aging and the shift in hormonal balance. FIG. 1 illustrates the cross-section of the male urethra in detail. The prostate is shown right below the bladder. The region of urethra surrounded by the prostate is the prostatic urethra, which is bounded by the bladder opening proximally and the membranous urethra distally, on the way to the cavernous (penile) urethra and external urethral orifice. As illustrated in FIGS. 2A and 2B, the prostatic urethra is compressed to a reduced diameter when the prostate is enlarged. This leads to the various symptoms observed in the progression of BPH, including but not limited to urinary frequency, urgency, nocturia, hesitancy, weak stream, straining, and prolonged voiding.

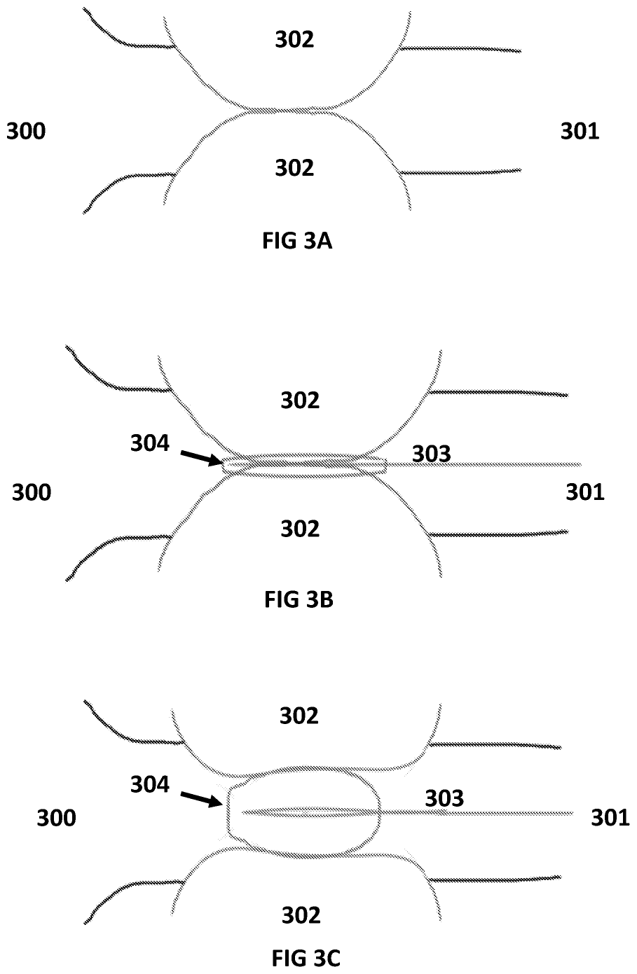

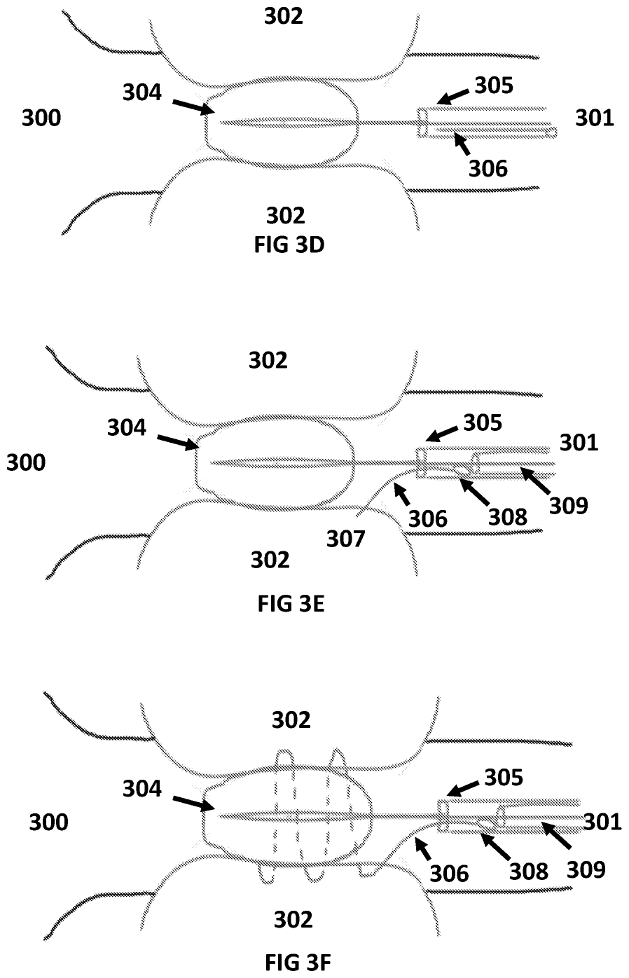

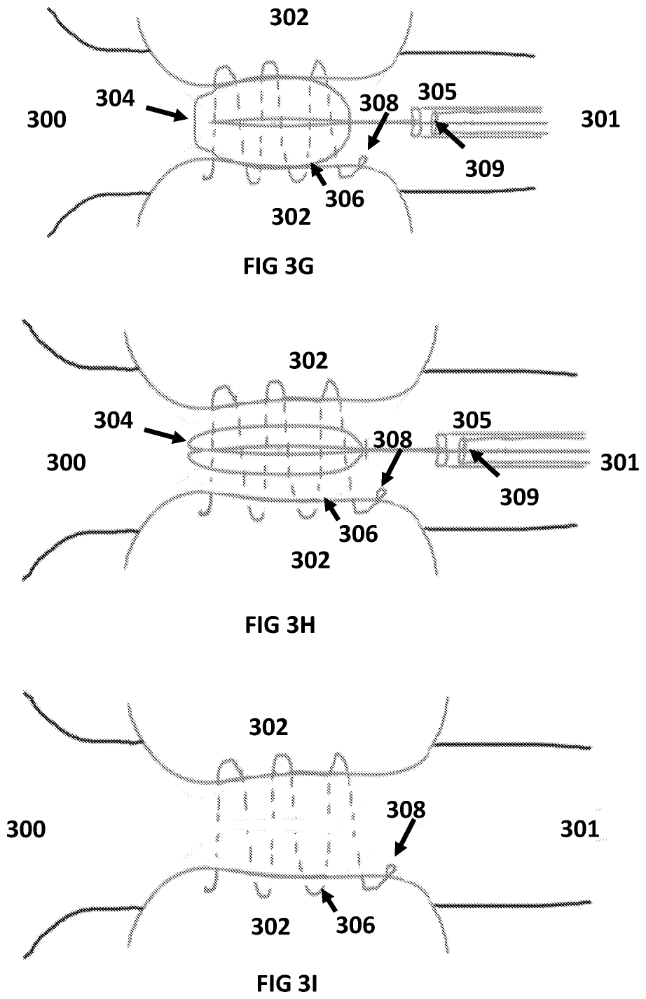

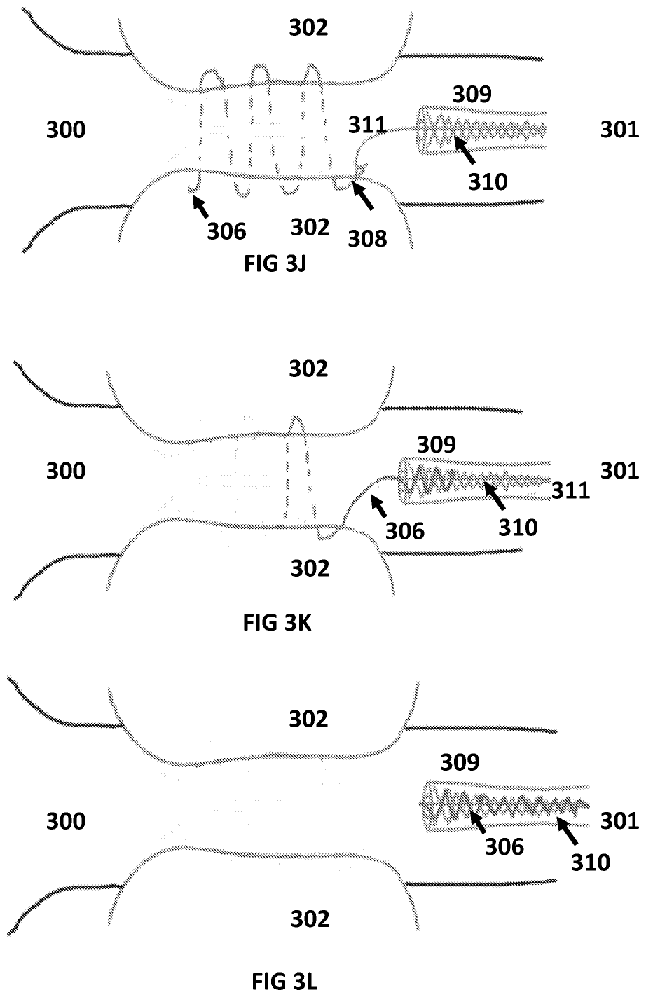

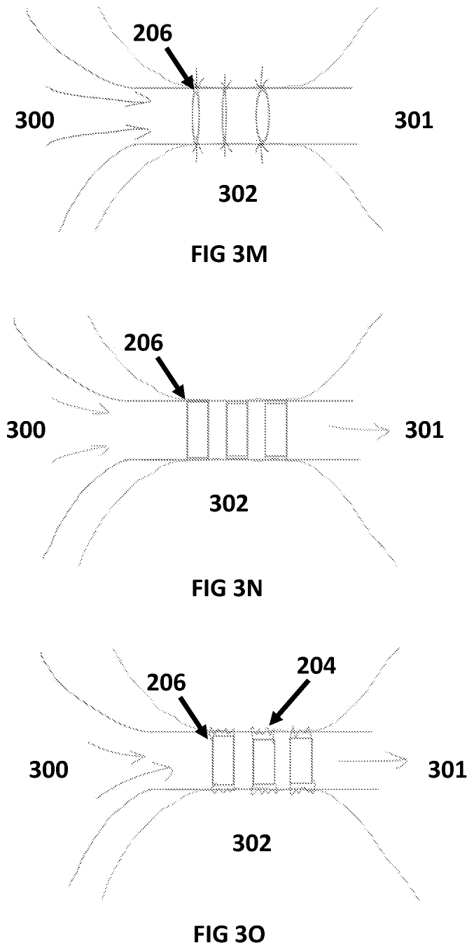

Disclosed herein are devices, including adjustable lumen tension rings (ALTRs) embodiments that can be configured to adjust the diameter and opening of the prostatic urethra. ALTRs can include various generally prosthetic devices, including tubular members configured to maintain or improve the patency of at least a portion of the urethra, such as the prostatic urethra. In some embodiments, a device can improve the patency of the prostatic urethra, but not the membranous urethra or penile urethra. In some embodiments, a device can be placed partially or completely within the membranous and/or penile urethra only, or in combination with the prostatic urethra to treat urethral strictures or stenosis that may not necessarily be caused by an enlarged prostate. FIG. 3A is a schematic side cross-sectional line drawing of an enlarged prostate 302 between the bladder side 300 and distal end of the urethra 301.

Disclosed herein are methods for deploying prosthetic devices, including fixed lumen or adjustable lumen tension rings (ALTRs) using an expandable member, such as a balloon technique, expandable device (e.g., movable cage with struts), or the like (FIGS. 3B to 3I). A guide catheter 303 with a balloon 304 is shown in FIG. 3B, which upon deployment and balloon expansion (e.g., with a gas, liquid, or other media) expands the prostatic urethra 302 via a circumferential radial outward force of the balloon as shown in FIG. 3C. An embodiment of an ALTR 306 can be implanted using a catheter or deployment device 305 into the prostatic urethra. In some embodiments, the leading edge of the ALTR 306 can optionally be used to create an incision in the prostatic urethral wall 307, as shown in FIGS. 3D and 3E. In some embodiments, the method is non-penetrating with respect to a urethral wall, such as the prostatic urethral wall 307. In some embodiments, the deployment of the ALTR 306 can be controlled by a spring-loaded plunger or threaded screw type tool 309. Some embodiments of the ALTR 306 can be deployed into the balloon 304 expanded prostate 302, such that it anchors or keeps the prostate expanded and away from the urethra, keeping the prostatic urethral space wide-open, as shown in FIG. 3F. Additionally, some embodiments of the ALTR may also include a manipulating feature 308 to allow further control, repositioning, and/or removal of the ALTR, as shown in FIGS. 3H and 3I. The expandable member e.g., balloon 304 can be deflated and removed once the ALTR 306 is implanted, as shown in FIG. 3H. FIG. 3H also illustrates the deflation of the balloon once the ALTR is able to anchor the prostate and keep the prostatic urethra wide-open. FIG. 3I illustrates the removal of the balloon and guide-wire and shows the ALTR expanding the prostate from outside the lumen and keeping the prostatic urethra wide-open. In some embodiments, the method is exclusively performed intra-urethrally. In some embodiments, the method does not involve circumferentially decreasing the diameter of one or both lobes of the prostate, e.g., with sutures.

Disclosed herein are manipulation features 308 within the prosthetic devices, such as ALTR 203. In some embodiments, the ALTR 306 can include one, two, or more manipulating features 308 such as barbs, grooves or loops to allow easy capture, re-alignment, re-positioning and removal of the ALTR 306, if/when needed. The manipulation features may be on or off axis, inside or outside the urethral wall, and penetrating or non-penetrating with respect to the urethral wall and/or prostate, for example. One key aspect of the manipulation feature in the ALTR in some embodiments is to allow control for reversibility of the procedure.

Disclosed herein are various methods of removing the prosthetic devices, including ALTR 306, in cases where reversibility or repositioning is desired. In some embodiments, a minimally invasive retrieval device 309 can be deployed via the urethra containing a retrieval wire with a feature 311 that links with the manipulating feature 308 in the ALTR 306, as shown in FIG. 3J. In some embodiments, the retrieval feature 311 and manipulating feature 308 can be linked via a hook-loop, hook-hook, or loop-hook type set-up, as shown in FIG. 3J. In some embodiments, one or both of the retrieval feature 311 and manipulating feature 308 can include complementary magnets, a gripper including, for example, movable jaws, an adhesive, a suction mechanism, and the like. In some embodiments, the retrieval device 309 may wind-in the ALTR 306 into a track or threaded feature 310 within the 309 devices, as shown in FIGS. 3K and 3L. The insertion, anchoring/connection to 308 and the removal of the ALTR 306 may all be performed by external controls (outside the body) of the 703 devices in some cases.

FIGS. 3M-3O illustrate additional embodiments of devices that can be configured to adjust the diameter and opening of the prostatic urethra. FIG. 3M is a side view of an embodiment of a lumen tension ring 206 implanted within the prostatic lumen, also illustrating the bladder side 300, distal end of the urethra 301, and prostate 302 as previously described. The device 206 can be in a circular or semi-circular wire/tube shape as shown in FIG. 3M. In some embodiments, the device 206 can be shaped as a cylinder or portion of a cylinder, cone, and similar variations as shown in FIG. 3N. The device 206 can also include one or more fixation elements 204 that can promote fixation of the device to the urethral wall to prevent migration and dislocation of the device 206. In some embodiments, the device 206 may include a protrusion or indentation for stabilizing and/or fixing the device at the wall. The fixation elements 204 can also include sub-elements to anchor the device to the wall, for example, grooves, teeth, ridges, or a saw-tooth pattern, for example, as described for example elsewhere herein.

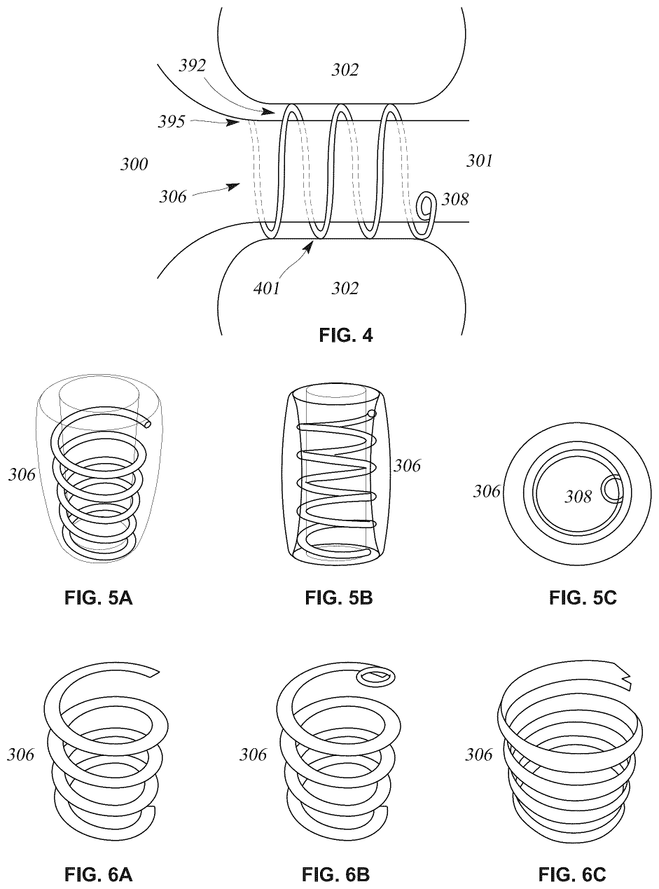

Disclosed herein are embodiments of prosthetic devices such as ALTR 306 such that the ALTR is inserted in the interstitial space 392 between the prostate 302 and the external urethral wall 395, keeping the prostate 302 separated from the wall 395 and thus the prostatic urethra wide-open for free flow of bodily fluids. In such embodiments, the ALTR does not penetrate the prostate itself. FIG. 4 illustrates a variant of the ALTR device where the device 306 is able to guide along the interstitial space 392 between the prostate 302 and the external urethral wall 395.

Disclosed herein are embodiments of prosthetic devices such as ALTR 306 in a wire form with a manipulating feature or features 308 at the proximal or distal end of the ALTR. FIGS. 5A, 5B, and 5C illustrates various views of such an embodiment. The manipulating feature can be, for example, an eyelet 308 extending radially inwardly as shown in FIG. 5C or outwardly in other embodiments, or other features as disclosed elsewhere herein.

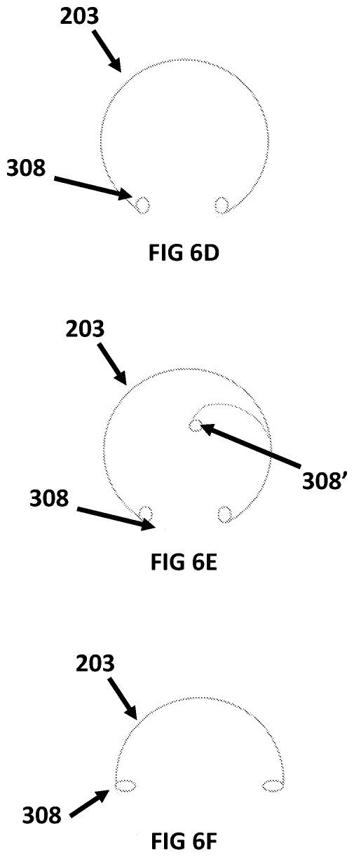

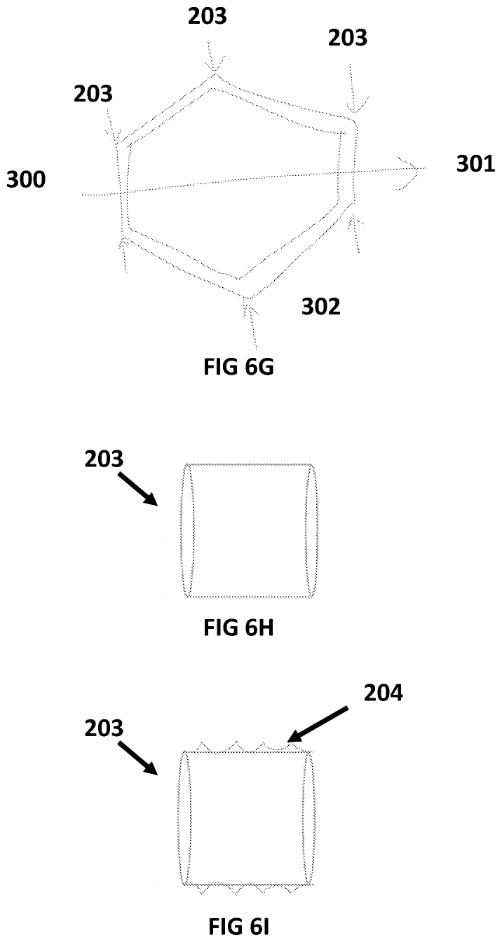

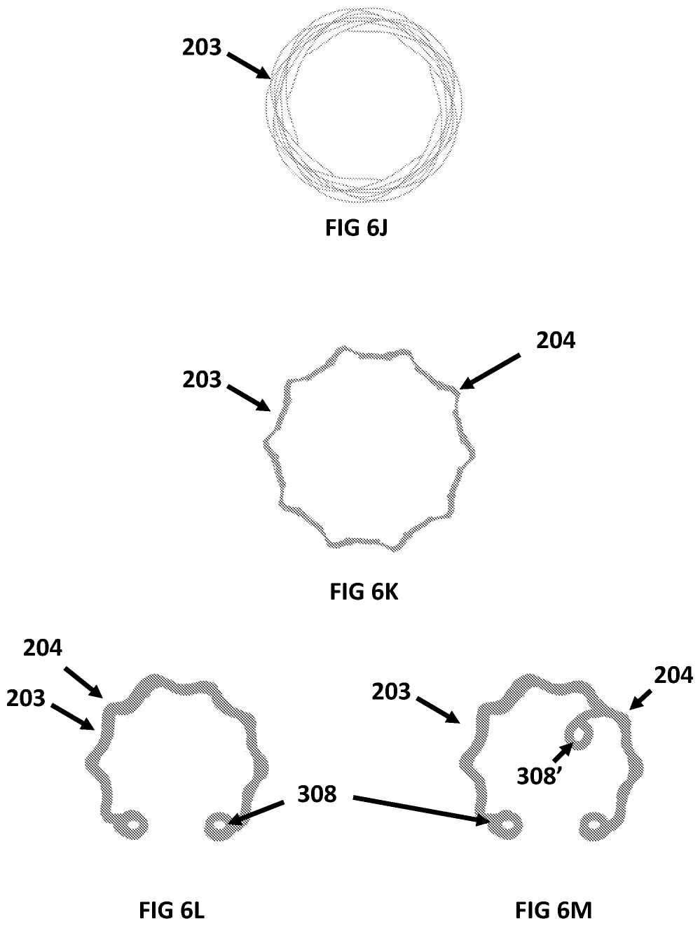

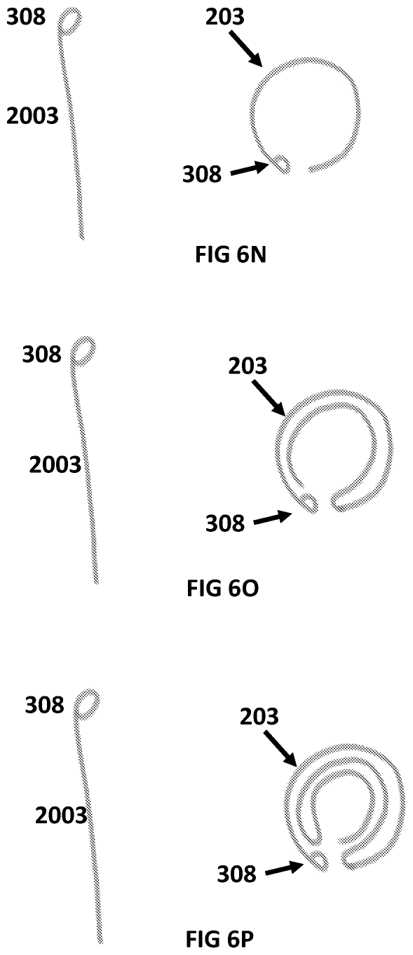

Disclosed herein are embodiments of prosthetic devices such as ALTR 306 in a flat or angulated ribbon form with a manipulating feature or features 308 at the proximal or distal end of the ALTR. For example, the structure could be generically helical with a plurality of revolutions as shown, with a flattened cross-section such as oval or rectangular for example. FIGS. 6A, 6B, and 6C illustrates various views of such an embodiment. The flat plane of the ribbon shown in the images (FIGS. 6A and 6B) is perpendicular to direction of travel. The ribbon can include proximal and/or distal free ends. Other variants of the embodiments could have the flat portion of the ribbon along the direction of travel, i.e. along the urethral wall or at various angles from the urethral wall. In some embodiments, the ribbon is a continuous structure where each revolution/loop of the ribbon is not connected to directly adjacent revolutions/loops at any points other than the winding of the continuous ribbon.

FIGS. 6D-F illustrate additional embodiments of devices that can include only a single, or a plurality of, complete or incomplete rings configured to maintain the patency of a lumen. A plurality of spaced-apart, directly adjacent, or overlapping discrete devices can be deployed in some embodiments. As disclosed for example elsewhere herein, a device 203 can include one or many manipulation features 308 such as grooves or loops to allow easy capture, re-alignment, re-positioning and removal of the device 203, if/when needed. FIG. 6E illustrates an embodiment of a device 203 with an additional manipulation feature 308′, that is off-axis and extends away from the wall. FIG. 6F shows an embodiment of a device 203 that is semi-circular in shape with a sweep angle of approximately 180 degrees. The sweep angle or central angle of the device 203 (or each sweep in devices that include a plurality of winds) can be between, for example, about 10 degrees and about 360 degrees, such as about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, or 360 degrees, or angles including any two of the foregoing angles.

Also disclosed herein are various sizings of devices 203 within the prostatic urethra or another body lumen. FIG. 6G illustrates use of multiple devices 203 of various sizes in the lumen to control the shape of the expansion and amount and direction flow. FIG. 6H illustrates a generally cylindrical embodiment a device 203 and FIG. 6I illustrates a cylindrical embodiment of a device 203 with fixation elements 204 that can provide anchoring to the urethral wall and extend radially outward as shown. In some embodiments, a device can include a constant sidewall. In some embodiments, a device does not include a constant sidewall or covering, and includes the wire outline form of the cylinder with the remaining area being free space open to the luminal sidewall.

Also disclosed herein are embodiments of devices 203 using a plurality of multifilaments or polyfilaments to expand the lumen wall (as shown in FIG. 6J). FIG. 6K illustrates an embodiment of the device 203 where fixation elements 204 such as irregular perimeter, crests & trough like features are used to provide anchoring and prevent migration. FIG. 6L and FIG. 6M illustrates another embodiment with manipulation features 308, 308′ that allow ease of manipulation, relocation, and retraction as described elsewhere herein.

Disclosed herein are embodiments of devices 203 that are delivered in a minimally invasive form 2003, such as a substantially linear biased form in some cases as shown, and retain the intended unbiased shape in-situ 203. FIG. 6N illustrates an embodiment of the device 203 where the sweep is slightly below 360 degrees, such as between about 300 degrees and about 360 degrees, or between about 300 degrees and about 359 degrees, or between about 320 degrees and about 359 degrees, and forming an incomplete loop. FIG. 60 illustrates an embodiment of the device 203 with multiple sweeps (spring-like) where the net sweep angle is between about 700 and about 740 degrees. FIG. 6P illustrates an embodiment of the device 203 with multiple sweeps (spring-like) where the net sweep angle is between about 1000 and about 1160 degrees. In some embodiments, the pathway of the shape of the device 203 generally reverse traces the shape of the first sweep. Depending on the sizing of the device 203, the number of sweeps, such as 360-degree sweeps can vary from 1 to 10 for example. Disclosed herein are embodiments that either partially or wholly cover the prostatic urethra. The illustrations shown here also demonstrate manipulation features 308 that allow ease of manipulation, relocation, and retraction.

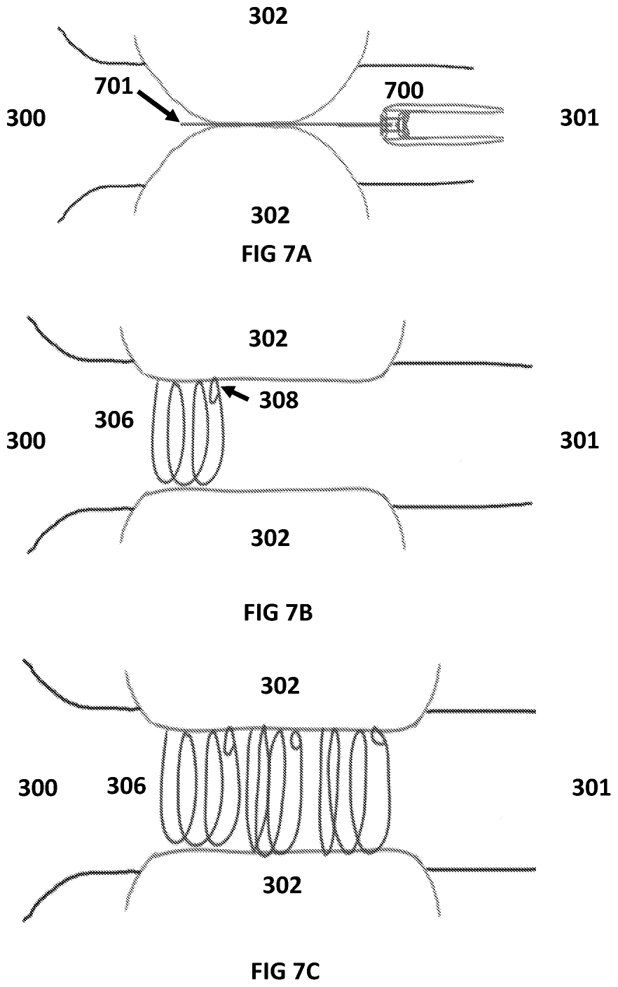

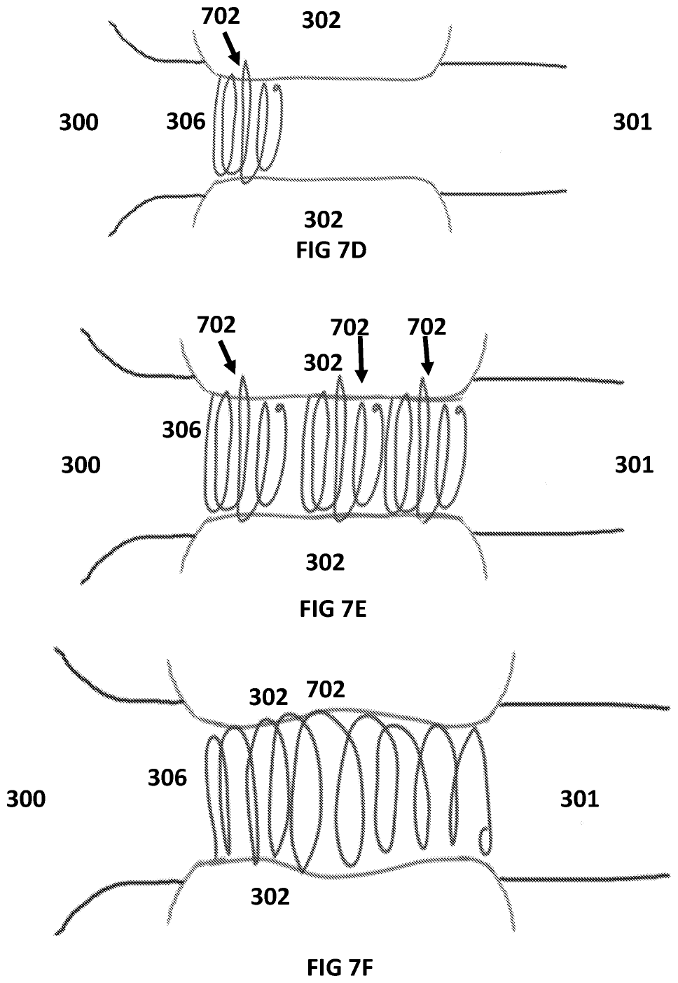

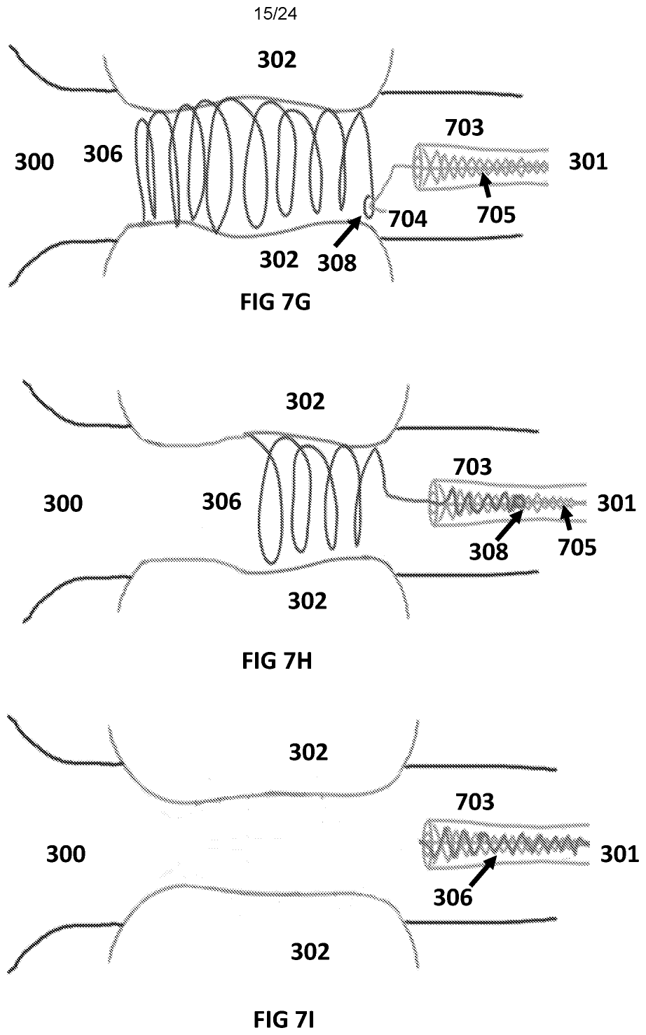

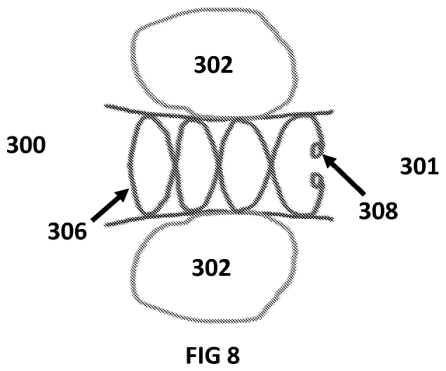

Disclosed herein are methods for deploying prosthetic devices, such as adjustable lumen tension rings (ALTRs) within the prostatic urethra (FIGS. 7B to 7F). In some embodiments, the leading edge 701 or other portion of the ALTR 306 can be inserted using an insertion device 700 between the prostatic overlap in the urethra, as shown in FIG. 7A. In some embodiments, upon partial or complete insertion, the ALTR 306 can spring into place, assuming a radially expanded configuration, and keep the prostatic urethra wide-open, due to the shape-memory nature of the ALTR 306 material, as shown in FIG. 7B. In some embodiments, multiple ALTRs 306 of similar or varying diameters can be deployed within the urethra depending on the prostatic anatomy, as shown in FIG. 7C. Some embodiments can include 2, 3, 4, 5, 6, 7, 8, 9, 10, or more devices, or ranges including any two of the foregoing values, such as between 1 and 10 devices, or between 2 and 8 devices for example. Each device can be placed directly adjacent to, e.g., in contact with each other, overlapping with each other, or spaced apart and not directly contacting each other, or combinations thereof. Additionally, some embodiments may have one, two, or more relatively larger diameter rings 702 within the ALTR 306 relative to other rings of the ALTR that are either centrally located, as shown in FIG. 7D or positioned elsewhere depending on the anchoring requirements within the prostatic urethra. In other embodiments, multiple such ALTRs 306 may be deployed within the urethra as shown in FIG. 7E. In some embodiments, one large ALTR could be deployed covering the entire length of the prostatic urethra, with similar or varying diameter along its length, as shown in FIG. 7F.

Disclosed herein are various methods of removing the prosthetic devices, such as ALTR 306, in cases where reversibility or repositioning is desired. In some embodiments, a minimally invasive retrieval device 703 can be deployed via the urethra containing a retrieval wire with a feature 704 that links with the manipulating feature 308 in the ALTR 306, as shown in FIG. 7G. In some embodiments, the retrieval feature 704 and manipulating feature 308 can be linked via a hook-loop, hook-hook, or loop-hook type set-up, as shown in FIG. 7G, or other features including those discussed elsewhere herein. In some embodiments, the retrieval device 703 may wind-in the ALTR 306 into a track or threaded feature 705 within the 703 devices, as shown in FIGS. 7H and 7I. The insertion, anchoring/connection to 308 and the removal of the ALTR 306 may all be performed by external controls (outside the body) of the 703 devices.

Disclosed herein are embodiments of prosthetic devices, including ALTR 306 that can form a double-helix or return pattern as shown in FIG. 8, in some cases with two discrete ends distally and a continuous loop end proximally without free ends. In some embodiments, there are two manipulating features 308 shown with both in the entry plane on the distal end of the device 306, although some embodiments could include only one, or three, four or more manipulating features for example.

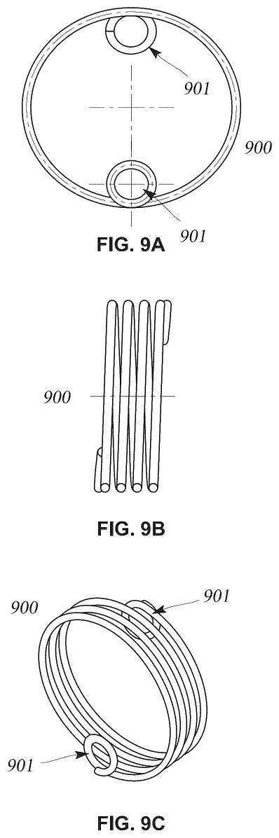

Disclosed herein are embodiments of prosthetic devices, including ALTR 306 that are configured to be delivered in a minimally invasive form and retain the intended shape in-situ 900. FIGS. 9A-C illustrates a variant of the ALTR with two manipulating features, such as eyelets or other features as disclosed herein, on both the leading and trailing ends. Each manipulating feature can be the same size and shape as shown, or different sizes and/or shapes in other embodiments. FIG. 9A is a top-view; FIG. 9B is a side view; and FIG. 9C is an isometric view of the ALTR. In some embodiments, the ALTR may be circular in shape with multiple sweeps (rotations). In some variants, ALTR 1000 may have 2 to 30 total sweeps (or rotations), whole or partial sweeps, such as 2, 3, 4, 5, 6, 7, 8, 9, 10, 12, 15, 20, 25, or 30 sweeps, or ranges including any two of the foregoing values. In some embodiments the pitch (separate between each ring) can be between about 0.0001″ and about 0.2″, such as about 0.0001″, 0.0005″, 0.001″, 0.005″, 0.01″, 0.05″, 0.1″, 0.2″, or ranges including any two of the foregoing values Disclosed herein are embodiments that either partially or wholly cover the prostatic urethra, such as for example, at least about, about, or no more than about 10%, 20%, 30%, 40%, 50%, 60%, 70%, 80%, 90%, 100% of the axial length of the prostatic urethra, or ranges including any two of the foregoing values The illustrations shown here also demonstrate the manipulation features 901 that allow ease of manipulation, relocation, and retraction using a separate retrieving device. In some embodiments, the implanted ALTR will not extend axially into the bladder and/or the membranous or penile urethra. In some embodiments, the implanted ALTR extends axially into one or more of the bladder, membranous, and/or penile urethra.

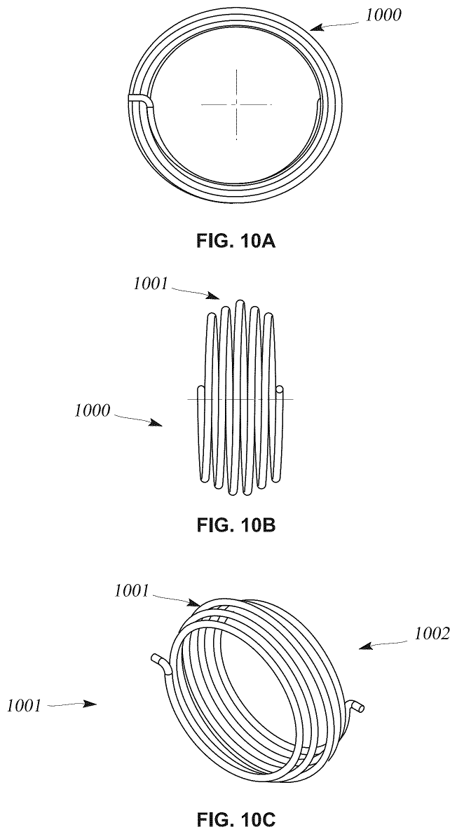

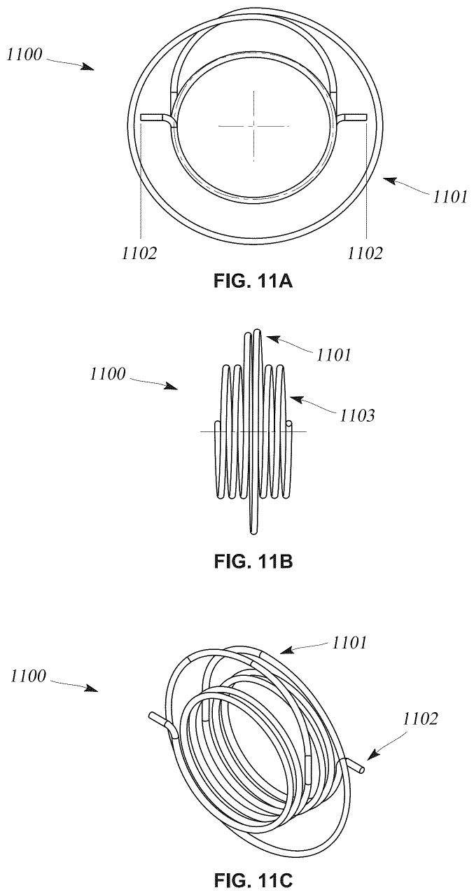

Disclosed herein are embodiments of prosthetic devices, including ALTR 306 that are delivered in a minimally invasive form and retain the intended shape in-situ 1000. FIGS. 10A-C illustrates a variant of the ALTR with variable diameter 1001 along the length of the ALTR. FIG. 10A is a top-view; FIG. 10B is a side view; and FIG. 10C is an isometric view of the ALTR. In some embodiments, the ALTR may be circular in shape with multiple sweeps (rotations). In some variants, ALTR 1000 may have 2 to 30 total sweeps (or rotations), whole or partial sweeps. In some embodiments the pitch (separation between each ring) within each section, or the entire device can be between about 0.0001″ to about 0.2″, such as about 0.0001″, 0.0005″, 0.001″, 0.005″, 0.01″. 0.05″. 0.1″ 0.15″, 0.2″, or ranges including any two of the foregoing values. Disclosed herein are embodiments that either partially or wholly cover the prostatic urethra. The illustrations shown here also demonstrate the manipulation features 1002 that allow ease of manipulation, relocation, and retraction using a separate retrieving device. In some embodiments, the central portion of the ALTR may have the largest diameter 1001 to allow better anchoring within the prostatic urethra and prevent migration within the urethra, with gradually decreasing diameters from the central portion to one or both ends as illustrated

Disclosed herein are embodiments of prosthetic devices, including ALTR 306 that can be delivered in a minimally invasive form and retain the intended shape in-situ 1100. FIGS. 11A-C illustrates a variant of the ALTR with a variable diameter 1101 along the length of the ALTR. FIG. 11A is a top-view; FIG. 11B is a side view; and FIG. 11C is an isometric view of the ALTR. In some embodiments, the ALTR may be circular in shape with multiple sweeps (rotations). In some variants, ALTR 1000 may have between about 2 and about 30 total sweeps (or rotations), whole or partial sweeps. In some embodiments the pitch (separate between each ring) can be between 0.0001″ to 0.2″, or ranges or values described elsewhere herein. Disclosed herein are embodiments that either partially or wholly cover the prostatic urethra. The illustrations shown here also demonstrate the manipulation features 1102 that allow ease of manipulation, relocation, and retraction using a separate retrieving device. In some embodiments, the central portion of the ALTR may have the largest diameter 1101 to allow better anchoring within the prostatic urethra and prevent migration within the urethra, but in contrast to FIGS. 10A-10C, the central portion of the ALTR can include 1, 2, 3, 4, 5, 6, 7, 8, 9, 10, or more (or ranges including any two of the foregoing values) relative constant larger diameter 1101 complete or partial sweeps or rotations, while the more lateral portions 1103 can have relatively constant diameter complete or partial sweeps or rotations that are of a smaller diameter than the central larger diameter sweeps or rotations. In some embodiments, the expanded length and/or number of rotations of the larger diameter central portion is about, or no more than about 50%, 40%, 30%, 20%, 10%, or less of that of the entire expanded length and/or number of rotations of the device.

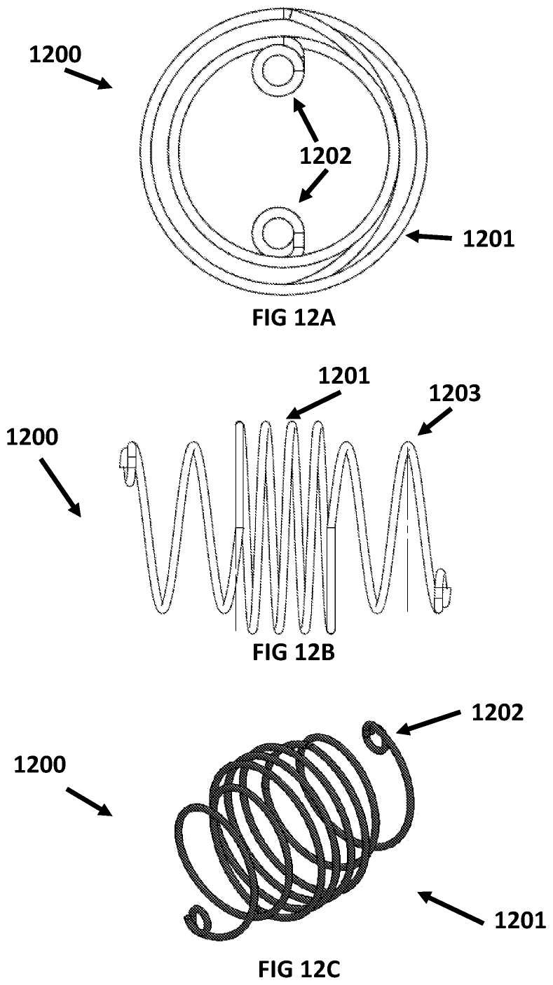

FIGS. 12A-C illustrate further embodiments of prosthetic devices, such as an ALTR that can incorporate features such as disclosed and illustrated in connection with, for example, FIGS. 11A-11C, with a larger expanded length/number of revolutions of the central portion 1201 relative to smaller diameter lateral portions 1203. The central portion 1201 can have a first, larger relatively constant diameter as shown, and the lateral portions 1203 can have a second, smaller relatively constant diameter, with a continuous inner lumen therebetween that can be as previously described. In other embodiments, either or both of the central or lateral portions can have gradual or stepped variable diameters. Manipulation features 1202, such as eyelets or other disclosed herein, can be attached, connected, or integrally formed at one or both ends of the device, or at other locations, and be made of the same, or different materials as the rest of the device itself. In some embodiments, the expanded length and/or number of rotations of the larger diameter central portion is about, or no more than about 50%, 60%, 70%, 80%, 90%, or more of that of the entire expanded length and/or number of rotations of the device.

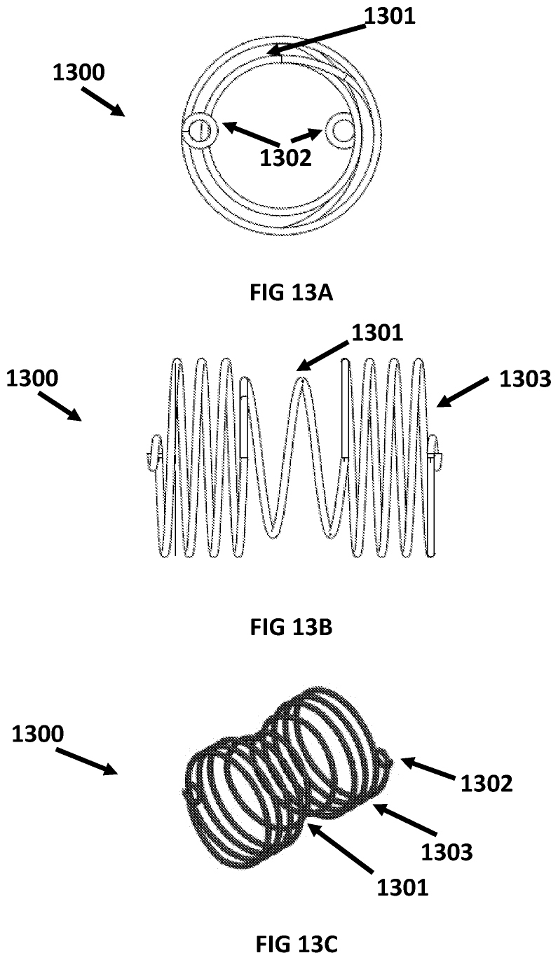

FIGS. 13A-C illustrate further embodiments of prosthetic devices, such as an ALTR that can incorporate features such as disclosed and illustrated in connection with, for example, FIGS. 11A-12C, except having with a smaller expanded length/number of revolutions of the central portion 1301 relative to larger diameter lateral portions 1303. The central portion 1301 can have a first, smaller relatively constant diameter as shown, and the lateral portions 1303 can have a second, larger relatively constant diameter, with a continuous inner lumen therebetween that can be as previously described. In other embodiments, either or both of the central or lateral portions can have gradual or stepped variable diameters. Manipulation features 1302, such as eyelets or other disclosed herein, can be attached, connected, or integrally formed at one or both ends of the device, or at other locations, and be made of the same, or different materials as the rest of the device itself. In some embodiments, the expanded length and/or number of rotations of the larger diameter central portion is about, or no more than about 50%, 60%, 70%, 80%, 90%, or more of that of the entire expanded length and/or number of rotations of the device.

In some embodiments, the larger diameter portions of the prosthetic devices can have an average or maximum diameter, for example, about or at least about 5%, 10%, 15%, 20%, 25%, 30%, 35%, 40%, 45%, 50%, 60%, 70%, 80%, 90%, 100%, or more relative to the average or maximum diameter of the smaller diameter portions, or ranges including any two of the foregoing values.

In some embodiments, a device can have a fully expanded/unbiased axial length of between about 5 mm and about 20 mm, or about, at least about, or no more than about 5, 6, 7, 8, 9, 10, 11, 12, 13, 14, 15, 16, 17, 18, 19, or 20 mm or more or less, or ranges including any two of the foregoing values.

In some embodiments, the larger diameter portions can have an outer diameter of about 8 mm to about 15 mm, or about, at least about, or no more than about 8, 9, 10, 11, 12, 13, 14, or 15 mm or more or less, or ranges including any two of the foregoing values.

In some embodiments, the smaller diameter portions can have an outer diameter of about 3 mm to about 10 mm, or about, at least about, or no more than about 3, 4, 5, 6, 7, 8, 9, 10 mm or more or less, or ranges including any two of the foregoing values.

In some embodiments, the central portion can have an axial length of between about 1 mm and about 15 mm or more or less, or about, at least about, or no more than about 1, 2, 3, 4, 5, 6, 7, 8, 9, 10, 11, 12, 13, 14, or 15 mm, or ranges including any two of the foregoing values.

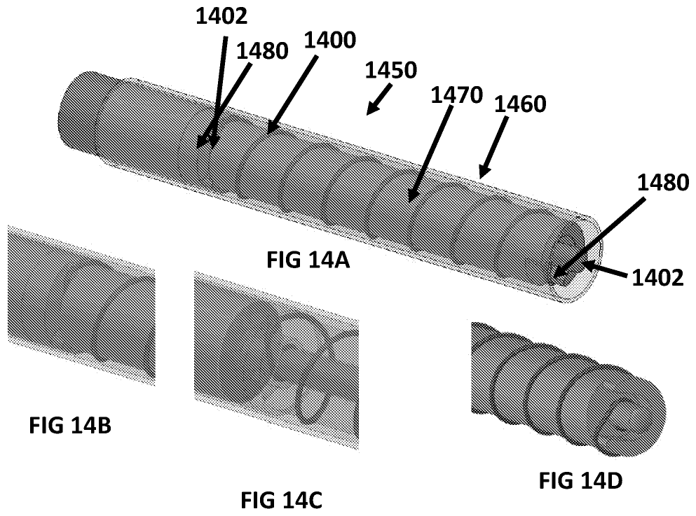

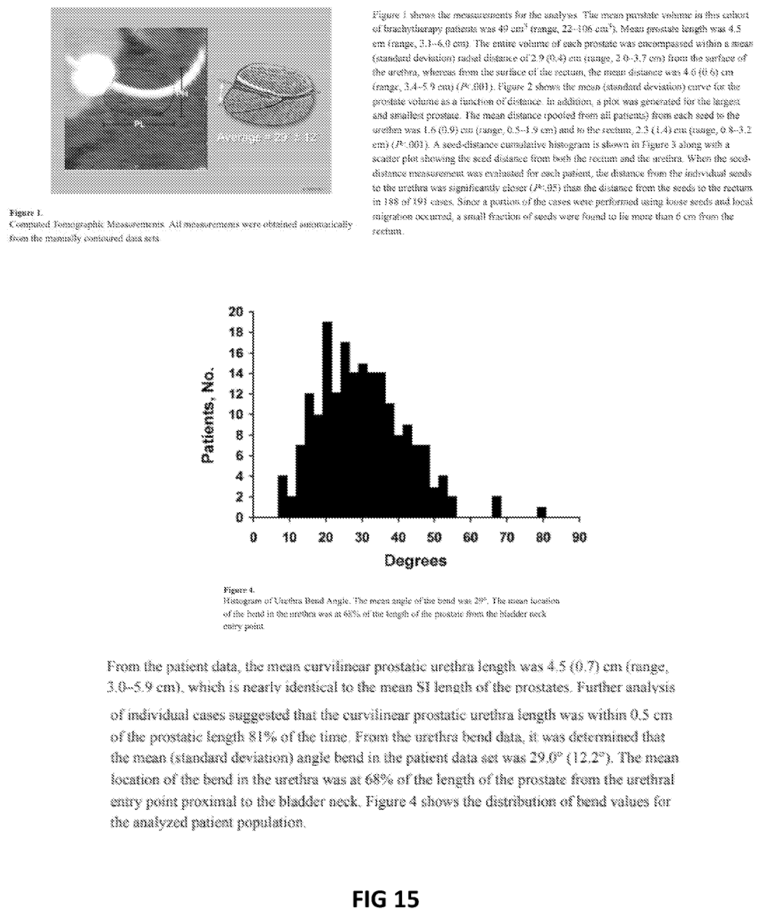

FIGS. 14A-14D schematically illustrate views of a distal end of a delivery system 1450 for a prosthetic device 1400. The delivery system 1450 can include a sheath 1460 and an inner member 1470 of which the prosthetic device 1400 such as an ALTR can wrap around its outer diameter in a relatively radially compressed configuration. The delivery system 1450 can be deployed to a desired anatomical location, such as the prostatic urethra, for example. An expandable member can be utilized to dilate the prostatic urethra as described elsewhere herein. The delivery system 1450 can have gripping or other locking features 1480 configured to reversibly lock or otherwise attach to corresponding manipulation features 1402 of the device. Upon removal of the sheath 1460 (or advancement of the inner member 1470) for example as shown in FIG. 14D and unlocking of the locking features 1480 as shown in FIG. 14C, the device 1400 can transform to its unbiased, radially expanded configuration.

Disclosed herein are embodiments of methods to deliver and/or retrieve the prosthetic devices, such as ALTR 306 in a minimally invasive form. Temperature of the ALTR 306 can be manipulated (e.g., increased or decreased) by an insertion tool whose temperature can be externally controlled through an energy source (electrical, mechanical, thermal, RF, ultrasonic, etc.), such that it can alter the shape (shrink or expand) of the ALTR 306 to make insertion or retrieval procedures both minimally invasive, responsive, and easy to manipulate/handle. In some embodiments, the device can be repositioned by at least initially torqueing (e.g., twisting) the device rather than axially pushing or pulling the device in a proximal or distal direction.

Disclosed herein are embodiments of methods to clean the prosthetic device, e.g., ALTR delivery 305/700 and retrieval 309/703 systems. Prior to deployment of the ALTR 306, a saline or other fluid flush may be performed to remove any urethral wall encrustation thereby improving the anchoring of the ALTR, and possibly minimizing the build-up of encrustation (e.g., from urinary sediment/crystal build-up on the device). An accessory to the ALTR 306 is a “roto rooter type” whereby a shaft is deployed within the lumen and a high-pressure saline jet(s) are sprayed unto an existing implant. The device can also optionally include a vacuum source to remove the encrustration-fluid combination. One objective of this accessory is to remove the encrustation of device thereby increasing the diameter of the urethra.

In some embodiments, a device can be configured to reduce undesirable encrustation with urine sediment, etc. that can build over time that can reduce the prostatic urethral diameter. In some embodiments, the device can include, for example, a wire, coil, or ribbon, for example with a relatively small diameter, that can be, for example, between about 0.005″ and about 0.02″, such as about or less than about 0.1″, 0.05″, 0.02″, 0.015″, or 0.01″ for example. In some embodiments, the device can have relatively larger diameter central portion or lateral portions, which sits further into the prostatic tissue behind the prostatic urethral luminal wall. Not to be limited by theory, one or more of the aforementioned features can limit exposure of the device to bodily fluids such as urine. In some embodiments, about or less than about 35%, 30%, 25%, 20%, 15%, 10% or less of the total surface area of the device, or ranges including any two of the foregoing values, is exposed to the flow of urine and potential encrustation when implanted. In other words, a majority of surface area of the device does not “see” flow of urine and as such be exposed to potentially undesirable encrustation. In some embodiments, the total surface area of the device exposed to urine is about or less than about 5.0, 4.5, 4.0, 3.5, 3.0, 2.5, 2.0, 1.5, 1.0, or less square centimeters, or ranges including any two of the foregoing values. In some embodiments, the length of the expanded device in situ (or the sum of multiple devices in situ) can be between about 1 cm and about 5 cm, such as about 1 cm, 2 cm, 3 cm, 4 cm, or 5 cm, or ranges including any two of the foregoing values, in some cases.

Disclosed herein are embodiments of methods to deliver the prosthetic device, e.g., ALTR 306 using the ALTR delivery 305/700 and retrieval 309/703 systems. The systems can either be manually powered or powered by an energy powered source whereby the deployment of the ALTR is controlled and motorized. At a predetermined force or power, the ALTR is forcefully deployed and anchored into the walls of the urethra.

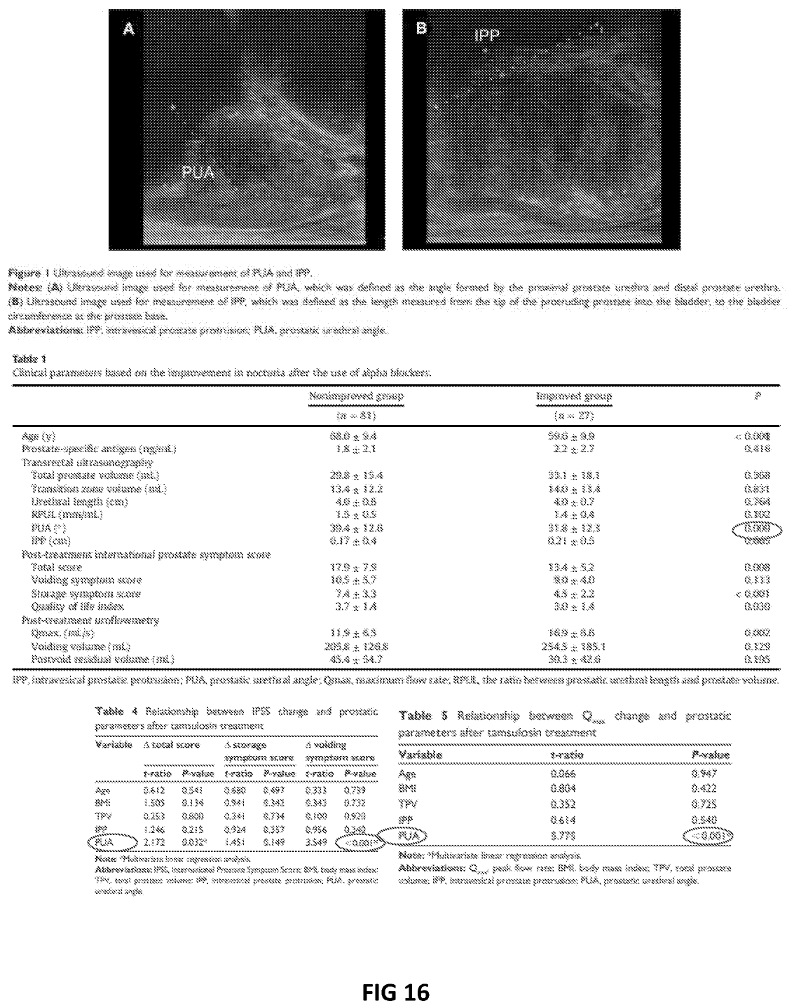

Disclosed herein are embodiments of methods to use pre-operative measurements of the prostatic urethral physiology to customize the device, e.g., ALTR for the specific requirement. Imaging techniques such as optical microscopy, ultrasonography, CT-scan, measurement of PUA (prostatic urethral angle), IPP (intravesical prostate protrusion), RPUL (the ratio between the prostatic urethral length and prostate volume), can be utilized, such as in a pre-treatment procedure to determine and customize the ALTR design to fit the specific physiological and anatomical need of the patient. One or a plurality of customized devices can then be manufactured and then implanted, e.g., in a separate procedure. However, the sizing procedure and implantation procedure can be combined into a single procedure in other embodiments. FIGS. 15 and 16 contain published information on clinical parameters based on prostatic urethral biometry (PUA, IPP, and RPUL, etc.) and correlation to clinical signs and symptoms of BPH.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “inserting the ALTR proximate to the distal end of the prostatic urethra” includes “instructing the inserting an ALTR proximate to the distal end of the prostatic urethra.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A device for maintaining patency of a prostatic urethra, comprising:
a single shape memory wire formed as an elongate coil and comprising a proximal end at a first end of the single shape memory wire, a distal end at an opposite, second end of the single shape memory wire, and a passageway between the proximal and distal ends configured to facilitate flow of body fluids between the proximal and distal ends, the elongate coil further comprising a plurality of partial or complete loops between the proximal end and the distal end, the elongate coil comprising a central coiled portion and lateral coiled portions along an axial length of the elongate coil, wherein the central coiled portion comprises a first diameter and the lateral coiled portions comprise a second diameter, wherein the first diameter is not equal to the second diameter, wherein loops of the elongate coil in the central coiled portion are longitudinally spaced apart from each other such that the loops avoid abutting one another, and
wherein the elongate coil further comprises a first radially compressed configuration transformable to a second radially enlarged configuration.

2. The device of claim 1, wherein the first diameter is larger than the second diameter.

3. The device of claim 1, wherein the first diameter is smaller than the second diameter.

4. The device of claim 1, wherein the central coiled portion has a generally constant first diameter throughout the entire length of the central coiled portion.

5-8. (canceled)

9. The device of claim 1, further comprising one or more manipulation features proximate at least one of the proximal end and the distal end.

10. (canceled)

11. The device of claim 9, wherein the one or more manipulation features are selected from the group consisting of an eyelet, a hook, a magnet, a threaded feature, and a loop.

12. (canceled)

13. (canceled)

14. The device of claim 1, wherein the proximal end comprises a sharp edge configured to pierce the urethral wall to anchor the elongate coil into the prostate and keep the elongate coil expanded from outside the urethral wall.

15-17. (canceled)

18. A delivery system comprising:
a device as in claim 1; and
a delivery tool comprising a device channel comprising an outer sheath and an inner member, the device configured to be disposed between the inner member and the outer sheath, the delivery tool comprising a locking feature configured to reversibly lock the device within the device channel.

19. The delivery system of claim 18, wherein the delivery tool comprises a cystoscope.

20. A method of treating benign prostatic hyperplasia in a patient, comprising:
expanding a lumen of the prostatic urethra in the patient using an expandable member;
radially expanding at least one device comprising a single shape memory wire formed as an elongate coil and comprising a plurality of windings along an axial length of the elongate coil, within the lumen of the prostatic urethra to expand a diameter of the prostatic urethra, wherein windings of the elongate coil are longitudinally spaced apart from each other such that the windings avoid abutting one another, the elongate coil comprising a larger diameter coiled portion along the axial length of the elongate coil and a smaller diameter coiled portion along the axial length of the elongate coil, the larger diameter coiled portion providing a radial force against the prostatic urethra sufficient to maintain patency of the prostatic urethra; and
unlocking a manipulation feature of the at least one device from a delivery tool.

21-25. (canceled)

26. The method of claim 20, wherein the delivery tool comprises a cystoscope comprising a device channel, an outer sheath, and an inner member, wherein the device is disposed between the inner member and the outer sheath and in a radially compressed configuration during delivery.

27. The method of claim 26, further comprising axially moving the outer sheath relative to the device to allow the device to radially expand.

28. The method of claim 26, wherein the device channel comprises a diameter of between about 1 mm and about 6 mm.

29. The method of claim 20, wherein following radially expanding the at least one device within the lumen of the prostatic urethra, no more than about 25% of an entire surface area of the device is exposed to urine flow within the prostatic urethra.

30. The method of claim 20, wherein following radially expanding the at least one device within the lumen of the prostatic urethra, no more than about 3 cm² of the entire surface area of the device is exposed to urine flow within the prostatic urethra.

31-33. (canceled)

34. The method of claim 20, wherein the at least one device is custom created based on biometry of the patient's prostatic urethra.

35. The method of claim 34, wherein the at least one device is custom created based on measured patient parameters selected from one or more of pre-operative imaging, prostatic dimensions, PUA (prostatic urethral angle), IPP (intravesical prostate protrusion), or RPUL (ratio between the prostatic urethral length and prostate volume).

36. A method of repositioning or removing a device for treating benign prostatic hyperplasia in a patient, comprising:
delivering an effector tool proximate the prostatic urethra and a previously implanted device residing within the lumen of the prostatic urethra, wherein the previously implanted device comprises a single shape memory wire formed as an elongate coil comprising a plurality of windings, wherein windings of the elongate coil are longitudinally spaced apart from each other such that the windings avoid abutting one another, the windings forming a lumen of the elongate coil, the lumen having a variable inner diameter, along an axial length of the elongate coil, the previously implanted device maintaining the patency of the lumen of the prostatic urethra;
contacting a manipulation feature of the previously implanted device with the effector tool;
locking the manipulation feature of the previously implanted device; and
removing or repositioning the previously implanted device.

37. The method of claim 36, further comprising delivering energy from the effector tool to the manipulation feature to change the size and/or shape of the previously implanted device.

38. The method of claim 36, wherein the manipulation feature comprises an eyelet, a hook, a loop, a magnet, or a threaded feature.

39. The device of claim 1, wherein the loops of the elongate coil in the central coiled portion are longitudinally spaced apart by a distance of 0.0001″ to 0.2″, 0.0001″, 0.0005″, 0.001″, 0.005″, 0.01″, 0.05″, 0.1″, or 0.2″.

40. The device of claim 1, wherein the single shape memory wire is configured to change from a first linear configuration to a second elongate coil configuration to form the elongate coil.

41. The device of claim 20, wherein the windings of the elongate coil are longitudinally spaced apart by a distance of 0.0001″ to 0.2″, 0.0001″, 0.0005″, 0.001″, 0.005″, 0.01″, 0.05″, 0.1″, or 0.2″.

42. The device of claim 20, wherein the single shape memory wire is configured to change from a first linear configuration to a second elongate coil configuration to form the elongate coil.

43. The device of claim 36, wherein the windings of the elongate coil are longitudinally spaced apart by a distance of 0.0001″ to 0.2″, 0.0001″, 0.0005″, 0.001″, 0.005″, 0.01″, 0.05″, 0.1″, or 0.2″.

44. The device of claim 36, wherein the single shape memory wire is configured to change from a first linear configuration to a second elongate coil configuration to form the elongate coil.
